(19) United States
(12) Patent Application Publication
(10) Pub. No.: US 2023/0226033 A1
(43) Pub. Date: Jul. 20, 2023

(54) STABLE FORMULATION OF EFINACONAZOLE

(71) Applicant: THE MATERIA COMPANY LIMITED, San Gwann, SGN (MT)

(72) Inventor: Matthaios VIDALIS, San Gwann (MT)

(21) Appl. No.: 18/007,945

(22) PCT Filed: Jun. 3, 2021
(86) PCT No.: PCT/EP2021/064943

§ 371 Date: Dec. 2, 2022

(30) Foreign Application Priority Data
Jun. 3, 2020 (MT) 4428

(51) Int. Cl. A61K 31/454; A61K 47/24; A61K 47/18; A61K 9/00

(52) U.S. Cl. CPC A61K 31/454; A61K 47/24; A61K 47/18; A61K 9/0014

(57) ABSTRACT

A new stable formulation comprising efinaconazole and its process for preparing. The formulation of the present invention exhibits low levels of impurities.

### FIELD OF THE INVENTION

The present invention relates to a formulation comprising the antifungal efinaconazole, which remains stable after storage for an extended period.

### BACKROUND OF THE INVENTION

Efinaconazole is a well-known pharmaceutical active ingredient used for the topical treatment of onychomycosis of the toenail(s) due to Trichophyton rubrum and Trichophyton mentagrophytes. Efinaconazole is prone to chemical degradation by oxidation and is hydrophobic due to its nature.

Formulations useful for the topical delivery of efinaconazole in the treatment of onychomycosis and other triazole antifungal drugs have been described in, e.g., U.S. Pat. Nos. 8,486,978 and 10,342,875. Said formulations comprise siloxanes which are suspected endocrine disruptors and reproductive toxins and comprise citric acid, also.

Thus, there is still a need to provide improved alternative efinaconazole formulations with great stability. The present inventors have come up with a stable formulation comprising efinaconazole which is prevents oxidation. It has now been surprisingly discovered that the above objective is met by providing a formulation comprising an acidifying agent selected from monoprotic acids, diprotic acids and a-keto-glutaric acid.

### SUMMARY OF THE INVENTION

The present invention relates to a new stable formulation comprising efinaconazole. In a first embodiment, the new formulation comprises efinaconazole, a volatile polar organic solvent, at least one phenolic antioxidant and an acidifying agent selected from monoprotic acids, diprotic acids, alpha-ketoglutaric acid, their pharmaceutically acceptable salts and mixtures thereof. Preferably, the acidifying agent is alpha-ketoglutaric acid.

In some embodiments the formulation is a solution or is a dispersion.

In some embodiments, the amount of efinaconazole is about 10% (w/w). In some embodiments, the amount of efinaconazole is in the range of about 8% (w/w) to about 12% (w/w).

In some embodiments, the volatile polar organic solvent is selected from ethyl acetate, 1-propanol, isopropanol, acetone and mixtures thereof. In some embodiments, the volatile polar organic solvent is ethyl acetate. In some embodiments the formulation comprises 45-85% of ethyl acetate, 1-propanol, isopropanol, acetone and mixtures thereof. In a preferred embodiment the formulation comprises 45-85% ethyl acetate.

In some embodiments, the formulation comprises a phenolic antioxidant. In some embodiments the formulation comprises 0.01%-5% phenolic antioxidant.

In some embodiments, the formulation comprises at least one chelating agent selected from ethylenediamine and pharmaceutically acceptable salts and mixtures thereof. The ethylenediamine and pharmaceutically acceptable salts is also a source of alkalinity.

In some embodiments, the formulation further comprises a co-solvent.

In some embodiments, the formulation is substantially free of siloxanes.

Embodiment 1. A formulation comprising efinaconazole, a volatile polar organic solvent, at least one phenolic antioxidant and an acidifying agent selected from monoprotic acids and diprotic acids, alpha-ketoglutaric acid, their pharmaceutically acceptable salts and mixtures thereof.

Embodiment 2. A formulation comprising efinaconazole, a volatile polar organic solvent, at least one phenolic antioxidant and an acidifying agent wherein the acidifying agent is alpha-ketoglutaric acid and pharmaceutically acceptable salts thereof.

Embodiment 3. A formulation according to Embodiment 1 comprising:
a. efinaconazole,
b. 45-85% volatile polar organic solvent
c. 0.01-5% phenolic antioxidant
d. an acidifying agent selected from monoprotic acids, diprotic acids, alpha-ketoglutaric acid and their pharmaceutically acceptable salts and mixtures thereof.
e. optionally, at least one chelating agent selected from ethylenediamine, and pharmaceutically acceptable salts and mixtures thereof
f. optionally, a co-solvent

Embodiment 4. A formulation according to Embodiments 1 to 2 wherein the formulation is a solution or a dispersion.

Embodiment 5. A formulation according to any one of Embodiments 1 to 3 wherein the volatile polar organic solvent selected from the group ethyl acetate, 1-propanol, isopropanol and acetone.

Embodiment 6. A formulation according to Embodiments 1 to 2 wherein the volatile polar organic solvent is 45-85% ethyl acetate.

Embodiment 7. A formulation according to Embodiment 1 wherein the phenolic antioxidant is butylated hydroxyanisole (BHA), butylated hydroxytoluene (BHT), β, γ, δ-tocopherol, dl-α-tocopherol, 2,6-di-tert-butyl-4-ethylphenol, 2,4-di-tert-butylphenol, 2,6-di-tert-butylphenol or mixtures thereof.

Embodiment 8. A formulation according to Embodiments 1 to 7 further comprising a phospholipid.

Embodiment 9. A formulation according to Embodiments 1 to 8, further comprising a co-solvent selected from glycerol, polyethylene glycols (PEGs), polypropylene glycol, water and mixtures thereof.

Embodiment 10. A formulation according to Embodiments 1 to 9, wherein the co-solvent is polypropylene glycol.

Embodiment 11. A process for preparing a formulation according to Embodiments 1 to 10 comprising:
i) Dissolving efinaconazole, and the phenolic antioxidant in solvent and optionally co-solvent
ii) Dissolve a chelating agent in mixture of step i) or in water
iii) Mixing solution or dispersion from step i) with the solution or dispersion of step ii).

Embodiment 12. A process for preparing a I formulation according to Embodiments 1-11 comprising:
i) Dissolving efinaconazole, and the phenolic antioxidant in ethyl acetate and optionally co-solvent
ii) Dissolve a chelating agent in mixture of step i) or in water
iii) Mixing solution or dispersion from step i) with the solution or dispersion of step ii).

### DETAILED DESCRIPTION OF THE INVENTION

The present invention relates to a new stable formulation comprising efinaconazole. In a first embodiment, the new formulation comprises efinaconazole, a volatile polar organic solvent, at least one phenolic antioxidant and an acidifying agent selected from monoprotic acids, diprotic acids, alpha-ketoglutaric acid, their pharmaceutically acceptable salts and mixtures thereof.

The term “stable” relates to a formulation capable of maintaining impurities profile below the ICH qualification thresholds, therefore acceptable stability with respect to the levels of impurities. Such stability is required for commercially relevant periods, for example about 6, 12, 18, or 24 months after manufacturing, during which times a product is stored in its original packaging at common ambient temperatures. Maintaining acceptable impurities level after opening is especially relevant for liquid or dispersions. Storage stability can be predicted by the impurity formation observed during storage for shorter times under “accelerated” stability testing conditions, such as at 40° C. and 75% relative humidity for 6 months.

The present invention relates to a new formulation which exhibit appreciable chemical storage stability. The embodiments exhibit pharmaceutically acceptable impurities profile below the ICH qualification thresholds, during accelerated stability testing. Some embodiments exhibit lower total impurities levels than the product used as reference product, marketed as Jublia® solution in USA.

The present formulation exhibit low total impurities level.

The term “efinaconazole” in the context of the present invention, refers to (2R ,3R)-2-and pharmaceutically acceptable salts thereof.

A chelating agent is a substance whose molecules can form several bonds to a single metal ion. A chelating agent is a multidentate ligand. Three widely used chelating agents are ethylenediamine, ethylenediaminetetraacetic acid and dimercaprol. According to the invention the chelating agent is selected from ethylenediamine, its pharmaceutically acceptable salts and mixtures thereof. Preferably ethylenediamine is used in some embodiments.

The term “antioxidants” in the context of the present invention, is compounds which scavenge free radicals by donating hydrogen to them, and they produce relatively stable antioxidant radicals with low standard reduction potential. Antioxidants slow down the oxidation rates by a combination of scavenging free radicals, chelating pro-oxidative metals, quenching singlet oxygen and photosensitizers. The higher stability of antioxidant radicals than the radicals in the finished product, is due to resonance delocalization throughout the phenolic ring structure. Preferably the bond dissociation energy for O—H of phenolic antioxidants is less than 80 kcal/mol. In a preferred embodiment the phenolic antioxidants are tocopherols, butylated hydroxytoluene (BHT), butylated hydroxyanisole (BHA), 2,6-di-tert-butyl-4-ethylphenol, 2,4-di-tert-butylphenol, 2,6-di-tert-butylphenol, tert-butylhydroquinone (TBHQ), propyl gallate (PG), lignans, flavonoids, and phenolic acids, ubiquinone (coenzyme Q), carotenoids, citric acid, amino acids and mixtures thereof. In a further preferred embodiment butylated hydroxytoluene (BHT), β, γ, δ-tocopherol, a-α-tocopherol (synthetic or natural form), 2,6-di-tert-butyl-4-ethylphenol, 2,4-di-tert-butylphenol, 2,6-di-tert-butylphenol and mixtures thereof are used.

The term “pharmaceutical acidifying agent” in the context of the present invention, is compounds which can create an acidic pH microenvironment within the formulation and therefore increase formulation stability. Preferably, the acidifying agent used are selected from monoprotic acids, diprotic acids and a-ketoglutaric acid. Their pharmaceutically accepted salts thereof can be also used. More preferably a-ketoglutaric acid is used. Monoprotic acids are those acids that are able to donate one proton per molecule during the process of dissociation (sometimes called ionization). Non-limiting examples of Zo monoprotic acids are hydrochloric acid (HCl), formic acid (HCOOH), acetic acid (CH₃COOH), nitric acid (HNO₃), benzoic acid (C₆H₅COOH), boric acid (H₃BO₃) and N-acetyl cysteine (NAC). Diprotic acids can donate two protons per acid molecule, in contrast to monoprotic acids that only donate one proton per molecule. Non-limiting examples of diprotic acids are succinic acid (CH₂)₂(CO₂H)₂, uric acid (C₅H₄N₄O₃), sulfuric acid (H₂SO₄), carbonic acid (H₂CO₃), oxalic acid (HOOCCOOH). Triprotic acids are able to donate three protons per acid molecule, in contrast to monoprotic acids that only donate one proton per molecule etc.

The present formulations are suitable to comprise at least one phospholipid, preferably phosphatidylcholine, more preferably at a concentration of 0.01 to about 10.0% (w/v).

Polar solvents decrease the radical scavenging activity of the antioxidants due to the intermolecular hydrogen bonding between oxygen or nitrogen in a polar solvent and OH group in phenolic antioxidants. Suitable pharmaceutically accepted volatile polar organic solvents are liquids such as esters, alcohols, ketones and saturated hydrocarbons with a high vapor pressure (greater than about 2 kPa at 20° C.) and boiling point less than about 100° C. at atmospheric pressure. Examples of suitable volatile polar organic solvents are ethyl acetate, butyl acetate, methyl acetate, 1-propanol, isopropanol (isopropyl alcohol), ethanol, acetone, methyl ethyl ketone and methyl isobutyl ketone. In a preferred embodiment the pharmaceutically accepted volatile polar organic solvents is chosen from ethyl acetate, 1-propanol, isopropanol, acetone and mixtures thereof. In a preferred embodiment the pharmaceutically accepted volatile polar organic solvents is ethyl acetate.

Co-solvents such as Glycerol, Polyethylene Glycols (PEGs), Propylene Glycol, Polypropylene Glycols, (PPGs), water and mixtures thereof, can be used. In a preferred embodiment Polypropylene Glycols (PPGs), water and mixtures thereof can be used.

In certain embodiments, the composition of the invention is free of volatile siloxanes. Siloxanes, also known as silicones, are cyclic and/or linear saturated cyclic and liner silicone-oxygen hydrides. Examples of cyclic siloxanes include polydimethylcyclosiloxanes, generally known as cyclomethicones (such as Zo cyclopentasiloxane, cyclotetrasiloxane, decylmethylcyclopentasiloxane, and the like). Examples of linear siloxanes include linear polysiloxanes (such as hexamethyldisiloxane, octamethyltrisiloxane, and the like).

In one embodiment the formulation comprises efinaconazole,
a. 45-85% a volatile polar organic solvent
b. 0.01-5% a phenolic antioxidant
c. an acidifying agent selected from monoprotic acids, diprotic acids, alpha-ketoglutaric acid, their pharmaceutically acceptable salts and mixtures thereof.
d. optionally, at least one chelating agent selected from ethylenediamine and pharmaceutically acceptable salts and mixtures thereof
e. optionally, a co-solvent

In another embodiment the formulation comprises efinaconazole,
a. 45-85% ethyl acetate
b. 0.01-5% a phenolic antioxidant
c. an acidifying agent selected from monoprotic acids, diprotic acids, alpha-ketoglutaric acid, their pharmaceutically acceptable salts and mixtures thereof.
d. at least one chelating agent selected from ethylenediamine and pharmaceutically acceptable salts and mixtures thereof
e. optionally, a co-solvent selected from the group glycerol, polyethylene glycols (PEGs), propylene glycol, polypropylene glycols (PPGs), water or mixtures thereof

In another embodiment the formulation comprises efinaconazole,
a. 45-85% volatile polar organic solvent, from the group ethyl acetate, 1-propanol, isopropanol and acetone
b. 0.01-5% phenolic antioxidant
c. a-ketoglutaric acid
d. ethylenediamine
e. optionally, 0.01-10% of at least one phosphatidylcholine
f. optionally, a co-solvent selected from the group glycerol, polyethylene glycols (PEGs), propylene glycol, polypropylene glycols (PPGs), water or mixtures thereof.

The term “reference product” refers to a currently or previously marketed efinaconazole solution, also described as the “originator” or “branded product” serving as a comparator in the studies. An “originator” or “branded” product are examples of a reference product. The preferred “reference product” is Jublia® solution marketed in USA.

### EXAMPLES

The formulation as described herein may be illustrated by the following examples which are not to be construed as limiting the scope of the invention: The “reference product” is Jublia® 10% Efinaconazole solution marketed in USA. Examples 1-4, formulated as shown in Table 1, contained 10% efinaconazole by weight. Examples 1-4 may optionally contain phospholipids and preferably phosphatidylcholine. The concentration (w/w %) of phosphatidylcholine may vary from 0.01%-10.00% by making known adjustments. Examples 1-4 were prepared according to the general manufacturing process:

Step a: In the appropriate amount of ethyl acetate, dissolve efinaconazole, polypropylene glycol 2000 and δ-tocopherol (Example 1) or α-tocopherol (synthetic or natural form) (Example 2) or Butylated Hydroxyanisole (Example 3) or 2,6-di-tert-butyl-4-ethylphenol and/or 2,4-di-tert-butylphenol and/or 2,6-di-tert-butylphenol (Example 4).

Step b: In another vessel, dissolve ethylenediamine and α-ketoglutaric acid in the appropriate amount of water.

Step c: Mix both solutions obtained by step a and step b until a clear, yellowish solution forms.

Alternatively, and if water is not used, dissolve ethylenediamine and α-ketoglutaric acid in the solution of step a. Skip step c.

TABLE 1
Pharmaceutical topical composition of Examples 1, 2, 3 and 4

| | Example 1 | Example 2 | Example 3 | Example 4 |
|---|---|---|---|---|
| Ingredients | (w/w %) | (w/w %) | (w/w %) | (w/w %) |
| Efinaconazole | 10 | 10 | 10 | 10 |
| Ethyl Acetate | 45-85 | 45-85 | 45-85 | 45-85 |
| δ-tocopherol | 0.10-5.00 | — | — | — |
| (δ-Vitamin E) | | | | |
| dl-α-tocopherol | — | 0.10-5.00 | — | — |
| (Vitamin E, synthetic | | | | |
| form) | | | | |
| Butylated | — | — | 0.10-2.00 | — |
| Hydroxyanisole (BHA) | | | | |
| 2,6-di-tert-butyl-4- | — | — | — | 0.10-2.00 |
| ethylphenol and/or | | | | |
| 2,4-di-tert-butylphenol | | | | |
| and/or 2,6-di-tert- | | | | |
| butylphenol | | | | |
| Optional: | 0.10-10.00 | 0.10-10.00 | 0.10-10.00 | 0.10-10.00 |
| Phosphatidylcholine | | | | |
| (PC - Phospholipon | | | | |
| 90 G) | | | | |
| Polypropylene Glycol | 5.00-30.00 | 5.00-30.00 | 5.00-30.00 | 5.00-30.00 |
| Mw 2000 (PPG 2000) | | | | |
| Ethylenediamine | 0.00001-0.01 | 0.00001-0.01 | 0.00001-0.01 | 0.00001-0.01 |
| (EDA) | | | | |
| α-ketoglutaric acid | 0.001-0.10 | 0.001-0.10 | 0.001-0.10 | 0.001-0.10 |
| Water, purified | 0-5 | 0-5 | 0-5 | 0-5 |

Comparative stability results are summarized in Table 2. The results after analysis for total impurity content of tested products: Reference product, Example 1, Example 2, Example 3 and Example 4 after 0, 3 and 6 months in accelerated conditions (40° C.±2° C., 75%±5% RH) are presented in the table below: Impurity results after 0, 3 and 6 months in accelerated conditions (40° C.±2° C., 75%±5% RH) Total impurities are acceptable since they are all below the ICH qualification thresholds. The reference product used in the comparative study is Jublia® marketed in USA.

TABLE 2
Comparative stability results

| Storage | Reference | | | | |
|---|---|---|---|---|---|
| Conditions | product | Example 1 | Example 2 | Example 3 | Example 4 |
| Accelerated | Total | Total | Total | Total | Total |
| Conditions | Impurities | Impurities | Impurities | Impurities | Impurities |
| at 40° C./ | | | | | |
| 75% RH | | | | | |
| t = 0 | 0.00% | 0.00% | 0.00% | 0.00% | 0.00% |
| 3 Months | 0.16% | 0.04% | 0.14% | 0.00% | 0.04% |
| 6 Months | 0.23% | 0.03% | 0.33% | 0.06% | 0.09% |

### Stability Tests Methods:

Impurity tests were determined by HPLC, using the below analytical method:

Column: Restek Roc C18 (150 mm×4.6 mm, 5 μm); injection volume: 25 μL; wavelength: UV, 260 nm; flow: 1 mL/min, column temperature: 30° C.

Buffer: 3.15 g of ammonium formate salt were dissolved in 1 L of water. pH adjusted to 4.5 with concentrated formic acid.

Mobile phase A: 100% Buffer

Mobile phase B: 100% Acetonitrile

| Time | | |
|---|---|---|
| (min) | MP A | MP B |
| 0 | 75 | 25 |
| 1.00 | 75 | 25 |
| 24.00 | 35 | 65 |
| 40.00 | 35 | 65 |
| 40.50 | 75 | 25 |
| 45.00 | 75 | 25 |

The diluent used for the samples' preparation is a 50:50 (v/v) solution of water:acetonitrile.

Thus, it should be appreciated that contemplated formulations will typically allow storage of the efinaconazole at least 6 months (and even longer) after first use without significant degradation of the efinaconazole. Efinaconazole may therefore be formulated for administration to human and various animals, and especially mammals. For example, formulations are suitable to be applied on the skin and the nail of mammals.

1. A formulation comprising efinaconazole, a volatile polar organic solvent, at least one phenolic antioxidant and an acidifying agent selected from monoprotic acids, diprotic acids, alpha-ketoglutaric acid, their pharmaceutically acceptable salts and mixtures thereof.

2. A formulation according to claim 1 further comprising at least one chelating agent selected from ethylenediamine and pharmaceutically acceptable salts thereof.

3. A formulation according to claim 1 comprising:
a. efinaconazole,
b. 45-85% volatile polar organic solvent
c. 0.01-5% phenolic antioxidant
d. an acidifying agent selected from monoprotic acids, diprotic acids, alpha-ketoglutaric acid,
e. at least one chelating agent selected from ethylenediamine and pharmaceutically acceptable salts thereof.
f. optionally, a co-solvent

4. A formulation according to any one of claims 1 wherein the volatile polar organic solvent selected from the group ethyl acetate, 1-propanol, isopropanol and acetone.

5. A formulation according to claim 1 wherein the volatile polar organic solvent is 45-85% ethyl acetate.

6. A formulation according to claim 1 to 5 wherein the phenolic antioxidant is butylated hydroxyanisole (BHA), butylated hydroxytoluene (BHT), β, γ, δ-tocopherol, α-tocopherol (synthetic or natural form), 2,6-di-tert-butyl-4-ethylphenol, 2,4-di-tert-butylphenol, 2,6-di-tert-butylphenol or mixtures thereof.

7. A formulation according to claim 6 further comprising a phospholipid.

8. A formulation according to claim 6, further comprising a co-solvent selected from glycerol, polyethylene glycols (PEGs), polypropylene glycol, water and mixtures thereof.

9. A formulation according to claim 8, wherein the co-solvent is polypropylene glycol.

10. A process for preparing a formulation according to claims 1 to 9 comprising:
i) Dissolving efinaconazole and the phenolic antioxidant in solvent and optionally co-solvent
ii) Dissolving an acidifying agent selected from monoprotic acids, diprotic acids, alpha-ketoglutaric acid, their pharmaceutically acceptable salts and mixtures thereof and optionally a chelating agent in mixture of step i) or in water
iii) Mixing solution or dispersion from step i) with the solution or dispersion of step ii).

11. A process for preparing a formulation according to claim 10 comprising:
i) Dissolving efinaconazole and the phenolic antioxidant in ethyl acetate and optionally co-solvent
ii) Dissolving a-ketoglutaric acid and a chelating agent in mixture of step i) or in water
iii) Mixing solution or dispersion from step i) with the solution or dispersion of step ii).
